SEQUENCE LISTING (60 sequences)

SEQ ID NO: 1 - DNA, 56 bp, Artificial Sequence
  Description of Artificial Sequence Synthetic primer
aatgatacggcgaccaccgagatctacactctttccctacacgacgctcttccgat

SEQ ID NO: 2 - DNA, 64 bp, Artificial Sequence
  Description of Artificial Sequence Synthetic primer
caagcagaagacggcatacgagatcactgtgtgactggagttcagacgtgtgctcttccgatct

SEQ ID NO: 3 - DNA, 49 bp, Artificial Sequence
  Description of Artificial Sequence Synthetic primer
cagacgtgtgctcttccgatctagatttcactctgaagatccggtccac

SEQ ID NO: 4 - DNA, 48 bp, Artificial Sequence
  Description of Artificial Sequence Synthetic primer
cagacgtgtgctcttccgatctagaaacagttccaaatcgmttctcac

SEQ ID NO: 5 - DNA, 46 bp, Artificial Sequence
  Description of Artificial Sequence Synthetic primer
cagacgtgtgctcttccgatctagcaagtcgcttctcacctgaatg

SEQ ID NO: 6 - DNA, 47 bp, Artificial Sequence
  Description of Artificial Sequence Synthetic primer
cagacgtgtgctcttccgatctaggccagttctctaactctcgctct

SEQ ID NO: 7 - DNA, 48 bp, Artificial Sequence
  Description of Artificial Sequence Synthetic primer
cagacgtgtgctcttccgatctagtcaggtcgccagttccctaaytat

SEQ ID NO: 8 - DNA, 47 bp, Artificial Sequence
  Description of Artificial Sequence Synthetic primer
cagacgtgtgctcttccgatctagcacgttggcgtctgctgtaccct

SEQ ID NO: 9 - DNA, 47 bp, Artificial Sequence
  Description of Artificial Sequence Synthetic primer
cagacgtgtgctcttccgatctagcaggctggtgtcggctgctccct

SEQ ID NO: 10 - DNA, 47 bp, Artificial Sequence
  Description of Artificial Sequence Synthetic primer
cagacgtgtgctcttccgatctagcaggctggagtcagctgctccct

SEQ ID NO: 11 - DNA, 47 bp, Artificial Sequence
  Description of Artificial Sequence Synthetic primer
cagacgtgtgctcttccgatctagagtcgcttgctgtaccctctcag

SEQ ID NO: 12 - DNA, 47 bp, Artificial Sequence
  Description of Artificial Sequence Synthetic primer
cagacgtgtgctcttccgatctagggggttggagtcggctgctccct

SEQ ID NO: 13 - DNA, 48 bp, Artificial Sequence
  Description of Artificial Sequence Synthetic primer
cagacgtgtgctcttccgatctaggggatccgtctccactctgamgat

SEQ ID NO: 14 - DNA, 48 bp, Artificial Sequence
  Description of Artificial Sequence Synthetic primer
cagacgtgtgctcttccgatctaggggatccgtctctactctgaagat

SEQ ID NO: 15 - DNA, 48 bp, Artificial Sequence
  Description of Artificial Sequence Synthetic primer
cagacgtgtgctcttccgatctaggggatctttctccaccttggagat

SEQ ID NO: 16 - DNA, 50 bp, Artificial Sequence
  Description of Artificial Sequence Synthetic primer
cagacgtgtgctcttccgatctagcctgacttgcactctgaactaaacct

SEQ ID NO: 17 - DNA, 46 bp, Artificial Sequence
  Description of Artificial Sequence Synthetic primer
cagacgtgtgctcttccgatctagcctcactctggagtctgctgcc

SEQ ID NO: 18 - DNA, 46 bp, Artificial Sequence
  Description of Artificial Sequence Synthetic primer
cagacgtgtgctcttccgatctagcctcactctggagtcmgctacc

SEQ ID NO: 19 - DNA, 49 bp, Artificial Sequence
  Description of Artificial Sequence Synthetic primer
cagacgtgtgctcttccgatctaggcagagaggctcaaaggagtagact

SEQ ID NO: 20 - DNA, 48 bp, Artificial Sequence
  Description of Artificial Sequence Synthetic primer
cagacgtgtgctcttccgatctaggaaggtgcagcctgcagaacccag

SEQ ID NO: 21 - DNA, 48 bp, Artificial Sequence
  Description of Artificial Sequence Synthetic primer
cagacgtgtgctcttccgatctaggaagatccagccctcagaacccag

SEQ ID NO: 22 - DNA, 46 bp, Artificial Sequence
  Description of Artificial Sequence Synthetic primer
cagacgtgtgctcttccgatctagtcgattctcagctcaacagttc

SEQ ID NO: 23 - DNA, 49 bp, Artificial Sequence
  Description of Artificial Sequence Synthetic primer
cagacgtgtgctcttccgatctagggagggacgtattctactctgaagg

SEQ ID NO: 24 - DNA, 46 bp, Artificial Sequence
  Description of Artificial Sequence Synthetic primer
cagacgtgtgctcttccgatctagttcttgacatccgctcaccagg

SEQ ID NO: 25 - DNA, 50 bp, Artificial Sequence
  Description of Artificial Sequence Synthetic primer
cagacgtgtgctcttccgatctagctgtagccttgagatccaggctacga

SEQ ID NO: 26 - DNA, 47 bp, Artificial Sequence
  Description of Artificial Sequence Synthetic primer
cagacgtgtgctcttccgatctagtagatgagtcaggaatgccaaag

SEQ ID NO: 27 - DNA, 49 bp, Artificial Sequence
  Description of Artificial Sequence Synthetic primer
cagacgtgtgctcttccgatctagtcctttcctctcactgtgacatcgg

SEQ ID NO: 28 - DNA, 44 bp, Artificial Sequence
  Description of Artificial Sequence Synthetic primer
cagacgtgtgctcttccgatctagaaccatgcaagcctgacctt

SEQ ID NO: 29 - DNA, 48 bp, Artificial Sequence
  Description of Artificial Sequence Synthetic primer
cagacgtgtgctcttccgatctagctccctgtccctagagtctgccat

SEQ ID NO: 30 - DNA, 49 bp, Artificial Sequence
  Description of Artificial Sequence Synthetic primer
cagacgtgtgctcttccgatctaggccctcacatacctctcagtacctc

SEQ ID NO: 31 - DNA, 43 bp, Artificial Sequence
  Description of Artificial Sequence Synthetic primer
cagacgtgtgctcttccgatctaggatcctggagtcgcccagc

SEQ ID NO: 32 - DNA, 42 bp, Artificial Sequence
  Description of Artificial Sequence Synthetic primer
cagacgtgtgctcttccgatctagattctggagtccgccagc

SEQ ID NO: 33 - DNA, 48 bp, Artificial Sequence
  Description of Artificial Sequence Synthetic primer
cagacgtgtgctcttccgatctagaactctgactgtgagcaacatgag

SEQ ID NO: 34 - DNA, 47 bp, Artificial Sequence
  Description of Artificial Sequence Synthetic primer
cagacgtgtgctcttccgatctagcagatcagctctgaggtgcccca

SEQ ID NO: 35 - DNA, 47 bp, Artificial Sequence
  Description of Artificial Sequence Synthetic primer
ctacacgacgctcttccgatctcttacctacaactgtgagtctggtg

SEQ ID NO: 36 - DNA, 47 bp, Artificial Sequence
  Description of Artificial Sequence Synthetic primer
ctacacgacgctcttccgatctcttacctacaacggttaacctggtc

SEQ ID NO: 37 - DNA, 47 bp, Artificial Sequence
  Description of Artificial Sequence Synthetic primer
ctacacgacgctcttccgatctcttacctacaacagtgagccaactt

SEQ ID NO: 38 - DNA, 45 bp, Artificial Sequence
  Description of Artificial Sequence Synthetic primer
ctacacgacgctcttccgatctaagacagagagctgggttccact

SEQ ID NO: 39 - DNA, 47 bp, Artificial Sequence
  Description of Artificial Sequence Synthetic primer
ctacacgacgctcttccgatctcttacctaggatggagagtcgagtc

SEQ ID NO: 40 - DNA, 44 bp, Artificial Sequence
  Description of Artificial Sequence Synthetic primer
ctacacgacgctcttccgatctcatacctgtcacagtgagcctg

SEQ ID NO: 41 - DNA, 43 bp, Artificial Sequence
  Description of Artificial Sequence Synthetic primer
ctacacgacgctcttccgatctccttcttacctagcacggtga

SEQ ID NO: 42 - DNA, 43 bp, Artificial Sequence
  Description of Artificial Sequence Synthetic primer
ctacacgacgctcttccgatctcttacccagtacggtcagcct

SEQ ID NO: 43 - DNA, 43 bp, Artificial Sequence
  Description of Artificial Sequence Synthetic primer
ctacacgacgctcttccgatctccgcttaccgagcactgtcag

SEQ ID NO: 44 - DNA, 41 bp, Artificial Sequence
  Description of Artificial Sequence Synthetic primer
ctacacgacgctcttccgatctagcactgagagccgggtcc

SEQ ID NO: 45 - DNA, 41 bp, Artificial Sequence
  Description of Artificial Sequence Synthetic primer
ctacacgacgctcttccgatctcgagcaccaggagccgcgt

SEQ ID NO: 46 - DNA, 43 bp, Artificial Sequence
  Description of Artificial Sequence Synthetic primer
ctacacgacgctcttccgatctctcgcccagcacggtcagcct

SEQ ID NO: 47 - DNA, 44 bp, Artificial Sequence
  Description of Artificial Sequence Synthetic primer
ctacacgacgctcttccgatctcttacctgtgaccgtgagcctg

SEQ ID NO: 48 - DNA, 44 bp, Artificial Sequence
  Description of Artificial Sequence Synthetic primer
ctacacgacgctcttccgatctctgaggagacggtgaccrkkgt

SEQ ID NO: 49 - DNA, 45 bp, Artificial Sequence
  Description of Artificial Sequence Synthetic primer
cagacgtgtgctcttccgatctagagagtcaccatgaccacagac

SEQ ID NO: 50 - DNA, 45 bp, Artificial Sequence
  Description of Artificial Sequence Synthetic primer
cagacgtgtgctcttccgatctagagagtcaccakkaccagggac

SEQ ID NO: 51 - DNA, 45 bp, Artificial Sequence
  Description of Artificial Sequence Synthetic primer
cagacgtgtgctcttccgatctagagagtcaccatgaccgaggac

SEQ ID NO: 52 - DNA, 45 bp, Artificial Sequence
  Description of Artificial Sequence Synthetic primer
cagacgtgtgctcttccgatctagagagtcaccattacyagggac

SEQ ID NO: 53 - DNA, 45 bp, Artificial Sequence
  Description of Artificial Sequence Synthetic primer
cagacgtgtgctcttccgatctagagagtcacgatwaccrcggac

SEQ ID NO: 54 - DNA, 45 bp, Artificial Sequence
  Description of Artificial Sequence Synthetic primer
cagacgtgtgctcttccgatctagagagtcaccatgaccaggaac

SEQ ID NO: 55 - DNA, 46 bp, Artificial Sequence
  Description of Artificial Sequence Synthetic primer
cagacgtgtgctcttccgatctagaccaggctcaccatywccaagg

SEQ ID NO: 56 - DNA, 44 bp, Artificial Sequence
  Description of Artificial Sequence Synthetic primer
cagacgtgtgctcttccgatctagggccgattcaccatctcmag

SEQ ID NO: 57 - DNA, 45 bp, Artificial Sequence
  Description of Artificial Sequence Synthetic primer
cagacgtgtgctcttccgatctagcgagtcaccatrtcmgtagac

SEQ ID NO: 58 - DNA, 44 bp, Artificial Sequence
  Description of Artificial Sequence Synthetic primer
cagacgtgtgctcttccgatctagcagccgacaagtccatcagc

SEQ ID NO: 59 - DNA, 46 bp, Artificial Sequence
  Description of Artificial Sequence Synthetic primer
cagacgtgtgctcttccgatctagagtcgaataaccatcaacccag

SEQ ID NO: 60 - DNA, 44 bp, Artificial Sequence
  Description of Artificial Sequence Synthetic primer
cagacgtgtgctcttccgatctaggacggtttgtcttctccttg

(19) United States
(12) Patent Application Publication
(10) Pub. No.: US 2020/0199650 A1
(43) Pub. Date: Jun. 25, 2020

(54) ANALYSIS SYSTEM FOR PERIPHERAL BLOOD-BASED NON-INVASIVE DETECTION OF LESION IMMUNE REPERTOIRE DIVERSITY AND USES OF SYSTEM

(71) Applicant: GENEPLUS-BEIJING, Beijing (CN)

(72) Inventors: Yuqi Wang, Beijing (CN); Yanfang Guan, Beijing (CN); Xin Yi, Beijing (CN); Tao Liu, Beijing (CN); Linjun Wu, Beijing (CN); Ling Yang, Beijing (CN)

(21) Appl. No.: 16/495,674

(22) PCT Filed: May 18, 2017
(86) PCT No.: PCT/CN2017/084799

§ 371 Date: Sep. 19, 2019

(51) Int. Cl. C12Q 1/686; G06F 17/18; C12N 15/11; C12Q 1/6881; C12Q 1/6869; G16B 30/00; G16B 35/00; G16B 20/20; G16B 20/30

(52) U.S. Cl. CPC C12Q 1/686; G06F 17/18; C12N 15/111; C12Q 1/6881; G16B 20/30; G16B 30/00; G16B 35/00; G16B 20/20; C12Q 1/6869

(57) ABSTRACT

A method for analyzing the diversity of immune repertoire of T cell receptors (TCR) or B cell receptors (BCR) of a cell-free DNA (cf-DNA) sample and of a nuclear DNA sample of peripheral blood mononuclear cell (PBMC), applicable in screening and determining the presence of lesion-infiltrating lymphocytes.

### TECHNICAL FIELD

The present invention pertains to a technical field of immune repertoire sequencing, and in particular relates to an analytical system for immune repertoire diversity of a T cell antigen receptor (TCR) or B cell antigen receptor (BCR) for a plasma cell-free DNA (cf-DNA) sample and a nuclear DNA (gDNA) sample of peripheral blood mononuclear cells (PBMCs) as well as applications thereof, thus screening and identifying the presence of lesions infiltrating lymphocytes (LILs).

### BACKGROUND ART

TCRs and BCRs are molecular structures that specifically recognize antigen peptides and mediate immune responses on the surface of lymphocytes, and are also among the most polymorphic regions in human genome. The diversity of lymphocyte receptor libraries directly reflects the diversity of immune responses of the body. The occurrence and development of different physiological processes and diseases can lead to changes in the state of related lymphocytes, and such changes make the best response and record for the occurrence and development of diseases. Consequently, research on immune repertoire of disease-specific lymphocytes has a very important role in revealing pathogeneses of diseases, developing therapeutic drugs, and judging therapeutic effects and prognoses of the diseases.

It is estimated that TCRs and BCRs in the same body can have a diversity of from 10¹¹ to 10¹². Such a huge diversity brings enormous potential to the body to bind to almost all “foreign” antigens, and it is such a diversity that plays a vital role in the maintenance of health. However, because of the limitations of the prior art and sampling, it is not possible for researchers to exhaust detection of all cells; besides, because of the existence of a large number of disease-unrelated lymphocytes, the researchers also have difficulties in understanding the analysis results of TCR or BCR immune repertoire. Moreover, lymphocytes move cyclically in the body and are colonized in various tissue structures; different pathogeneses cause different immune responses, and also cause different types of disease-specific lymphocytes to appear in different tissue structures, and therefore it is often difficult to obtain a representative sample. In general, lymphocytes colonized in lesion sites are mainly lesions infiltrating lymphocytes (LILs), and therefore obtaining pathological tissues of lesion sites by biopsy has certain guiding significance. However, because of the limitation of sampling and the heterogeneity of the lesion sites, single tissue sampling cannot fully represent all the characteristics of a disease. Therefore, it is of vital importance to develop a simple, timely, accurate and non-invasive screening method for LILs.

### CONTENTS OF INVENTION

Our findings show that there are a large number of nucleic acid fragments derived from lymphocyte apoptosis in cf-DNA; in normal human plasma, a frequency of lymphocyte-derived TCR/BCR rearrangement genes obeys a normal distribution; however, in a patient's body, since an immune response is activated, apoptosis occurs in a large number of disease-related lymphocytes or lesions infiltrating lymphocytes (LILs), resulting in a skewed TCR/BCR gene rearrangement frequency distribution. Therefore, outliers in a skewed distribution are just the specific TCRs/BCRs from lesion sites of diseases. In the present invention, we can just find out TCR/BCR gene clones of lesions infiltrating lymphocyte (LILs), by performing a comparative analysis of immune repertoire of TCRs/BCRs in a cfDNA sample and its corresponding PBMC gDNA sample, and removing interference of a lymphocyte total library in PBMCs using a filtering method developed by us. Accordingly, we obtain an analysis of TCR/BCR immune repertoire diversity based on peripheral blood cf-DNA and PBMCg DNA samples, and actualize non-invasive screening and identification of lesions infiltrating lymphocytes.

Specifically, the present invention provides a method for analyzing immune repertoire diversity of TCRs or BCRs in cell-free DNA (cf-DNA) samples in plasma as well as gDNA samples isolated from peripheral blood mononuclear cells (PBMCs), and actualizes effective screening and identification of lesions infiltrating lymphocytes.

More specifically, the method includes the following steps:

I. Constructing a reference sequence set and designing a specific amplification primer according to a TCR or BCR reference sequence.

II. Preparation of samples

1. Drawing 10 mL of peripheral blood of a subject to be tested, storing in an EDTA anticoagulation tube, followed by separating plasma and then using Ficoll lymphocyte separation solution to complete the separation of peripheral blood mononuclear cells (PBMCs);

2. Extracting a cf-DNA from a plasma sample, and extracting a nuclear DNA from a PBMC sample; and

3. determining DNA quality.

III. Library preparation and sequencing

1. PCR1: subjecting cf-DNA and PBMC-DNA samples to multiplex PCR amplification of CDR3 sequences of a TCR β chain and a BCR H chain, respectively;

2. Magnetic bead purification: purifying an amplification product of the previous step;

3. PCR2: further amplifying a target fragment using a library linker primer;

4. Fragment purification: performing purification and fragment selection on an amplification product;

5. Library quantification and quality control; and

6. sequencing using a high-throughput sequencer.

IV. Analyze off-line data by bioinformatics

1. Bioinformatics analysis of immune repertoire: performing MiXCR software analysis, filtering out low-quality data, correcting PCR and sequencing errors, and identifying CDR3 sequences;

2. performing Non-invasive lesions infiltrating lymphocytes analysis (NILILa), including the following steps:

if the ranking of relative abundance of N TCRs/BCRs in plasma constitutes a collection Y(y₁≤y₂≤ . . . ≤y_{N}), since a normal TCR/BCR library in a patient's plasma comes from a normal distribution population, the disease-specific TCR/BCR sub-library released from his lesion sites will cause a skewed distribution of a plasma TCR/BCR total library after entering plasma. Supposing a probability density function of his skewed distribution is cdf: F(Y|θ), wherein θ is the decision parameter set of F. θ can be obtained by solving Equation 1 based on the principle of minimum variance. Equation 1 is described as follows:

$$θ = arg {min}_{θ} {\sum }_{i ∈ Λ}^{ } {[ g ( {y}_{i} ) - g ( {F}^{- 1} ( {F}_{i} \vert θ ) ) ]}^{2} ,$$

wherein A is an index set of Y subset, yᵢ represents a relative abundance of the i^{th} TCR/BCR CDR3, g is a monotonic function that can be differentiated within the value range of Y, and arg mi_{ƒ}min f (θ) refers to a value corresponding to θ when an objective function ƒ(θ) takes a minimum value. This equation is solved to get cdf, the expression of cdf being as follows

$$\frac{1}{2} + \frac{1}{2} erf \{ \frac{{( y - μ )}^{2}}{\sqrt{2} σ} \} ,$$

in this equation erf(θ) is an error function, wherein μ is a mean and σ is a variance. A TCR/BCR frequency distribution detected in plasma can be determined according to this model probability density distribution function. Supposing there are two thresholds [symbol] _{ρ}^{±}, when a frequency of TCR/BCR is higher than [symbol] _{ρ}⁺ or lower than [symbol] _{ρ}⁻, the number of CDR3 is ρ_{±}, and Equation 2 is solved, the expression of Equation 2 being as follows:

$${𝓁}_{ρ}^{\pm } = {F}^{- 1} ( {δ}_{\pm } ∓ \frac{{ρ}_{\pm }}{N} \vert θ ) ,$$

δ_{±} in the equation refers to a standard deviation, and therefore a threshold [symbol] _{ρ}^{±} is obtained as follows:

$${𝓁}_{ρ}^{\pm } = \sqrt{2} σ {erf}^{- 1} [ \pm ( 1 - 2 \frac{{ρ}_{\pm }}{N} ) ] + μ ,$$

furthermore, in order to explore more outlier TCRs/BCRs associated with lesion sites, ρ_{±} is set to 1, a relative abundance value [symbol] _{ρ}⁺ characterizing outlier TCRs/BCRs is calculated, and then this value can be used as a boundary of distinguishing outliers, and a frequency value corresponding to this point is called plasma B (boundary, B) point;

furthermore, in order to avoid an impact of a lymphocyte total library in PBMCs on results, the filtering method shown in FIG. 2 is used to eliminate interference of the lymphocyte total library in the PBMCs: an abscissa is an order of a frequency of clones detected in PBMCs from high to low, and an ordinate is an order of a frequency of clones detected in plasma from low to high; in this chart, frequency coordinates of each clone in two samples are marked, and then two points are found: abscissa and ordinate values of the first point are both maximum values, and the second point has an abscissa value of 0 and an ordinate value of B value. These two points are connected to form a line segment which divides coordinates into two parts: the upper right part is a distribution area of lesions infiltrating lymphocytes, and the lower left part is a distribution area of other background clones. Points in the upper right part are output, and are just CDR3 sequences of lesions infiltrating lymphocytes.

In addition, the present invention further relates to a bioinformatics analysis unit comprising executing the following instructions:

1) performing MiXCR software analysis, filtering out low-quality data, correcting PCR and sequencing errors, and identifying CDR3 sequences;

2) performing Non-invasive lesions infiltrating lymphocytes analysis (NILILa), comprising the following steps:

if the ranking of relative abundance of N TCRs/BCRs in plasma constitutes a collection Y(yᵢ≤y₂≤ . . . ≤y_{N}), since a normal TCR/BCR library in a patient's plasma comes from a normal distribution population, the disease-specific TCR/BCR sub-library released from his lesion sites will cause a skewed distribution of a plasma TCR/BCR total library after entering plasma; supposing a probability density function of the skewed distribution is cdf: F(Y|θ), wherein θ is the decision parameter set of F; θ can be obtained by solving Equation 1 based on the principle of minimum variance, Equation 1 being described as follows:

$$θ = arg {min}_{θ} {\sum }_{i ∈ Λ}^{ } {[ g ( {y}_{i} ) - g ( {F}^{- 1} ( {F}_{i} \vert θ ) ) ]}^{2} ,$$

wherein A is an index set of Y subset, yᵢ represents a relative abundance of the i^{th} TCR/BCR CDR3, and g is a monotonic function that can be differentiated within the value range of Y; this equation is solved to get cdf of which the expression is as follows:

$$\frac{1}{2} + \frac{1}{2} erf \{ \frac{{( y - μ )}^{2}}{\sqrt{2} σ} \} ,$$

a TCR/BCR frequency distribution detected in plasma can be determined according to this model probability density distribution function; supposing there are two thresholds [symbol] _{ρ}^{±}, when a frequency of TCR/BCR is higher than [symbol] _{ρ}⁺ or lower than [symbol] _{ρ}⁻, the number of CDR3 is ρ_{±}, and Equation 2 is solved, the expression of Equation 2 being as follows:

$${𝓁}_{ρ}^{\pm } = {F}^{- 1} ( {δ}_{\pm } ∓ \frac{{ρ}_{\pm }}{N} \vert θ ) ,$$

a threshold [symbol] _{ρ}^{±} is obtained as follows:

$${𝓁}_{ρ}^{\pm } = \sqrt{2} σ {erf}^{- 1} [ \pm ( 1 - 2 \frac{{ρ}_{\pm }}{N} ) ] + μ ,$$

furthermore, in order to explore more outlier TCRs/BCRs associated with lesion sites, ρ_{±} is set to 1, a relative abundance value [symbol] _{ρ}^{±} characterizing outlier TCRs/BCRs is calculated, and then this value can be used as the boundary of distinguishing outliers, and a frequency value corresponding to this point is called plasma B (boundary, B) point;

furthermore, in order to avoid an impact of a lymphocyte total library in PBMCs on results, the following chart is drawn to exclude interference from the lymphocyte total library in PBMCs: an abscissa is an order of a frequency of clones detected in PBMCs from high to low, and an ordinate is an order of a frequency of clones detected in plasma from low to high; in this chart, frequency coordinates of each clone in two samples are marked, and then two points are found: abscissa and ordinate values of the first point are both maximum values, and the second point has an abscissa value of 0 and an ordinate value of B value; these two points are connected to form a line segment which divides coordinates into two parts: the upper right part is a distribution area of lesions infiltrating lymphocytes, and the lower left part is a distribution area of other background clones; points in the upper right part are output, and are just CDR3 sequences of lesions infiltrating lymphocytes.

The present invention further relates to a hardware device such as a computer that runs the above-mentioned bioinformatics analysis unit.

### DESCRIPTION OF DRAWINGS

FIG. 1: Amplification primer sequences of a CDR3 region of a TCR β chain.

FIG. 2: Amplification primer sequences of a CDR3 region of a BCRH chain.

FIG. 3: Detection results of a NILILa method: the points distributed in the upper right part of the slash are the screened LILs.

### SPECIFIC MODES FOR CARRYING OUT THE INVENTION

### EXAMPLE 1

### Construction of TCR Libraries of Peripheral Blood and Tumor Tissue Samples from Patients

The tumor tissue g-DNA samples, the peripheral plasma cf-DNA samples and the g-DNA samples of PBMCs from 3 patients with malignant tumors were extracted and were subjected to sequencing detection of TCR β chain CDR3; the specific operations and results are as follows:

Sample List:

TABLE 1
List of cases and samples

| Case No. | Lymphocyte subpopulation | Library No. |
|---|---|---|
| Case 1 | plasma cf-DNA | Lab-A-1 |
| | g-DNA of PBMC sample | Lab-A-2 |
| | g-DNA of tumor tissue sample | Lab-A-3 |
| Case 2 | plasma cf-DNA | Lab-B-1 |
| | g-DNA of PBMC sample | Lab-B-2 |
| | g-DNA of tumor tissue sample | Lab-B-3 |
| Case 3 | plasma cf-DNA | Lab-C-1 |
| | g-DNA of PBMC sample | Lab-C-2 |
| | g-DNA of tumor tissue sample | Lab-C-3 |

Sampling and Processing of Tumor Tissue and Peripheral Blood Samples

1) Plasma separation: 2 tubes (5 mL/tube) of peripheral blood of a subject were extracted and placed in an EDTA anticoagulation tube, the tube was gently turned upside down (preventing cell rupture) 6-8 times for sufficient mixing; the following processing was carried out within 4-6 hours of the day of blood collection: the blood was centrifugated at 1600 g for 10 minutes at 4° C., and the supernatant (plasma) was divided into a plurality of 1.5 mL/2 mL centrifuge tubes after centrifugation, and the intermediate layer leukocytes should not be pipetted during the pipetting; after centrifugation at 16000 g for 10 minutes at 4° C. to remove residual cells, the supernatant (plasma) was transferred to a new 1.5 mL/2 mL centrifuge tube, during which process leukocytes at the bottom of the tube should not be pipetted, that is, the required plasma after separation was obtained. After the plasma sample was processed, the separated plasma was stored in a −80° C. refrigerator for later use, and repeated freezing and thawing should be avoided.

2) PBMC separation: 4 volumes of sterile physiological saline was added to the remaining blood cells, and turned upside down to mix them; 3 ml of the cellular layered liquid was placed in a 15 ml centrifuge tube, and 4 ml of the diluted whole blood cells were carefully pipetted and superimposed on the layered liquid surface along the tube wall, which was performed using multiple tubes in case of a volume of larger than 4 ml. After centrifugation at 400 g for 30 minutes at room temperature, the lymphocyte layer was carefully pipetted, placed into another centrifuge tube, added with 5 or more volumes of sterile physiological saline, and centrifuged at 400 g for 10 minutes at room temperature; afterwards, the supernatant was discarded, PBS was added, and a cell suspension was obtained by gentle blow and set aside.

3) Tumor tissue sample processing: a tumor tissue block after surgery was washed with sterile physiological saline, and a soybean-sized tissue block was cut out at a portion where the tumor cell content was high. Then the tissue block was divided into two parts, one of which was sent to a pathological lab to detect the tumor cell content, and the other was quickly soaked into the prepared RNAlater, stored for 12 hours at room temperature, and then stored at −20° C. for later use. If the pathological test reveals that the tumor cell content is greater than 70% and the necrotic tissue content is less than 20%, the sample is qualified and the next test is conducted.

Extraction and Quality Control of Sample Nucleic Acids

1) Plasma cf-DNA extraction: plasma cf-DNA extraction was performed fully in accordance with the extraction kit instructions of QIAamp Circulating Nucleic Acid Kit (Qiagen). After the extraction was completed, the concentration of the extracted DNAs was quantified using Qubit (the Quant-iT™ dsDNA HS Assay Kit, Invitrogen), and the distribution of fragments of the extracted DNAs was detected using Bioanalyzer 2100 (Agilent).

2) G-DNA extraction of PBMC samples: extraction was performed fully in accordance with the extraction kit instructions of QIAGEN QIAamp DNA Mini Kit. After the extraction was completed, the concentration of the extracted DNAs was quantified using Qubit (the Quant-iT™ dsDNA HS Assay Kit, Invitrogen), and the distribution of fragments of the extracted DNAs was detected using Bioanalyzer 2100 (Agilent).

3) G-DNA extraction of tumor tissue samples: extraction was performed fully in accordance with the extraction kit instructions of QIAGEN QIAamp DNA Mini Kit. After the extraction was completed, the concentration of the extracted DNAs was quantified using Qubit (the Quant-iT™ dsDNA HS Assay Kit, Invitrogen), and the distribution of fragments of the extracted DNAs was detected using Bioanalyzer 2100 (Agilent).

Library Construction

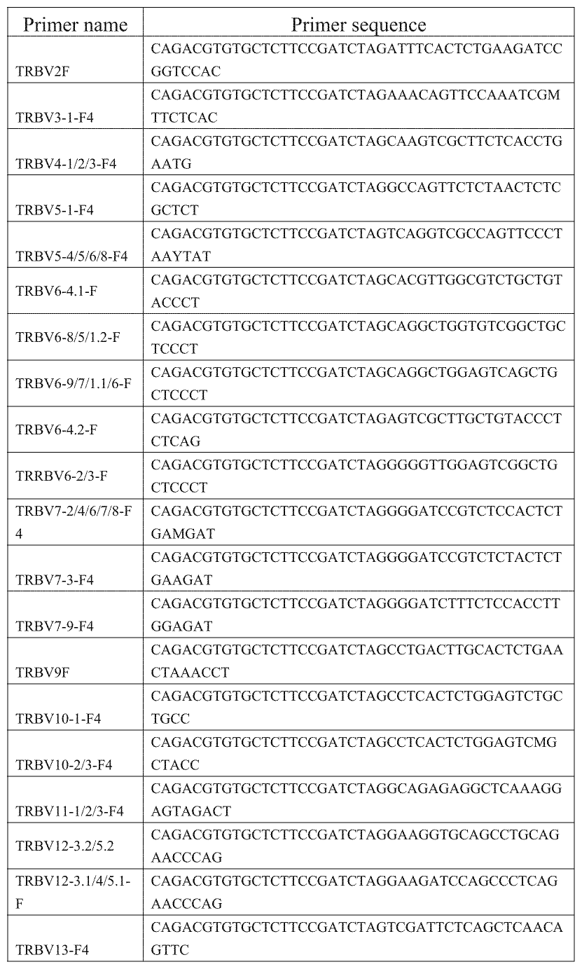

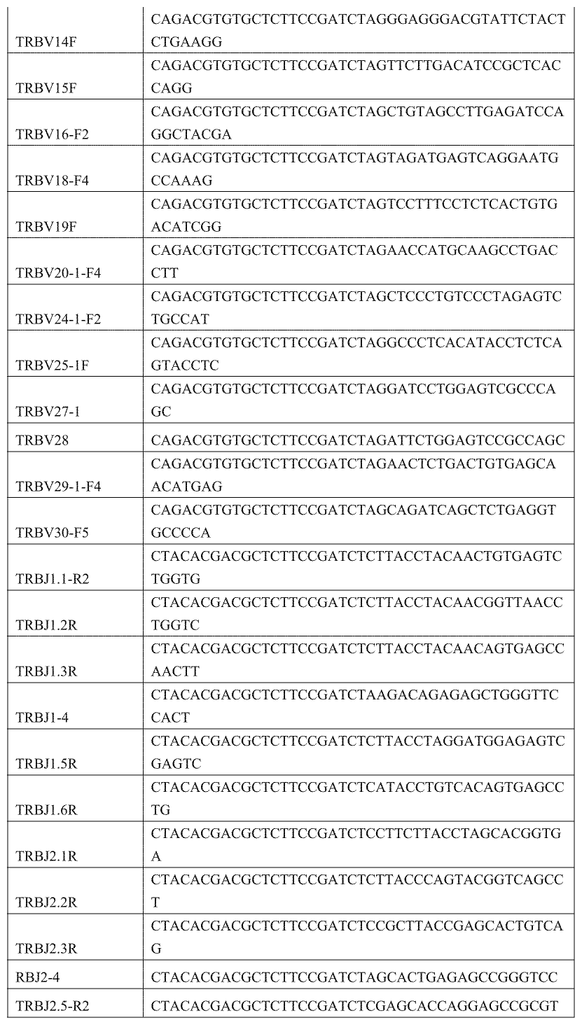

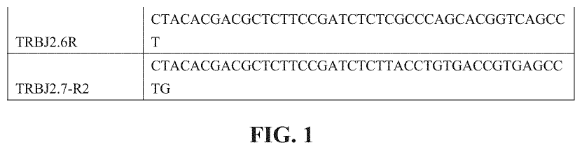

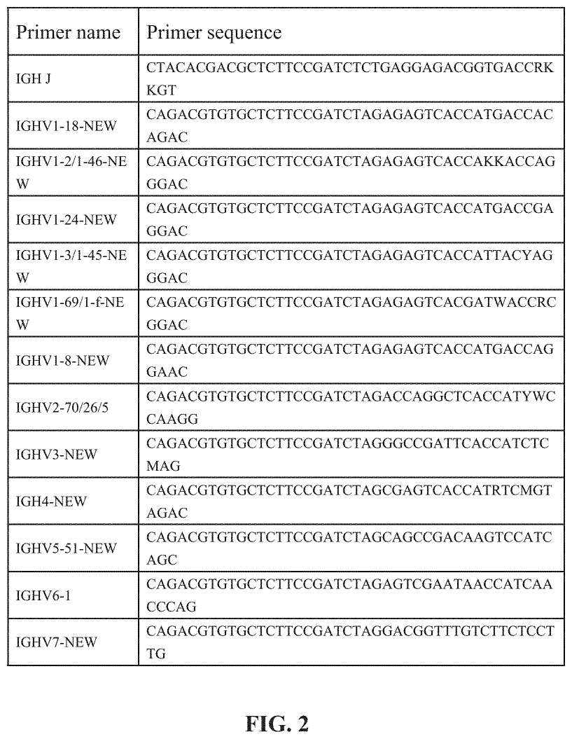

PCR1 amplification: a CDR3 region of a TCR β chain was amplified by TCR-specific primers, using a kit of QIAGEN Multiplex PCR Kit (Qiagen), the primer sequences being shown in FIG. 1. The sequences of the specific primers for amplifying a BCR H chain CDR3 region is shown in FIG. 2. The reaction system is shown in Table 2:

TABLE 2
PCR1 reaction system

| Components | Volume (μL) |
|---|---|
| 2 × QIAGEN Multiplex | 25 μL |
| 5 × Q solution | 5 μL |
| Primer working fluid | 2 μL |
| Sample DNA | X μL |
| NF-H₂O | Supplemented to |
| | 50 μL |
| Total Volume | 50 μL |

Conditions for multiplex PCR amplification were as follows: pre-denaturation at 95° C. for 15 min; denaturation at 94° C. for 30 s, annealing at 60° C. for 90 s, extension at 72° C. for 30 s, which were carried out for a total of 10 cycles; final extension at 72° C. for 5 min; maintained at 4° C.

2) Magnetic bead purification: the PCR reaction mixture was transferred to one 1.5 mL centrifuge tube, and the amplified sample was purified using an AMPure XP DNA Purification kit (SPRI beads).

3) PCR2 amplification: an Illumina common primer and an Index primer were used to amplify products of the previous step, and the kit of KAPA HiFi PCR Kits (kapabiosystems) was used for operation; the reaction system is shown in Table 3:

TABLE 3
PCR2 reaction system

| Components | Volume |
|---|---|
| Purified DNA | 23 μL |
| Primer1 common (10 uM) | 1 μL |
| Primer Index_ 5 (10 uM) | 1 μL |
| 2 × KAPA hifi hot start Master Mix | 25 μL |
| Total Volume | 50 μL |

Conditions for PCR amplification were as follows: pre-denaturation at 98° C. for 1 min; denaturation at 98° C. for 20 s, annealing at 65° C. for 30 s, extension at 72° C. for 30 s, which were carried out for a total of 28 cycles; final extension at 72° C. for 5 min; maintained at 4° C.

wherein the sequence of Primer 1 common primer is:

AATGATACGGCGACCACCGAGATCTACACTCTTTCCCTACACGACGCTC
TTCCGAT;

Index 5 primer (Primer Index_5) is:

CAAGCAGAAGACGGCATACGAGATCACTGTGTGACTGGAGTTCAGAC
GTGTGCTCTTCCGATCT.

4) Magnetic bead purification: a PCR reaction mixture was transferred to one 1.5 mL centrifuge tube, and the amplified sample was purified using an AMPure XP DNA Purification kit (SPRI beads).

5) 2% agarose gel recovery: a gel for TCR was cut to recover a target fragment of 250-350 bp in length. The fragment was dissolved in NF—H₂O having a volume of 30 uL and stored, and then the library construction was completed.

Library Quality Control Detection

The quality control of DNA fragments and concentrations of a library was carried out by Bioanalyzer 2100 (Agilent).

Sequencing

A NextSeq500 (Illumina) PE151+8+151 program was used for sequencing, and sequencing experiments carried out sequencing operations in accordance with the manufacturer's instructions.

### EXAMPLE 2

### Bioinformatics Analysis of Immune Repertoire

1. After the quality control of the data generated by the sequencing was passed, the analysis was performed according to the public software MiXCR (https://mixcr.readthedocs.org/en/latest/index.html).

Sequences obtained by the sequencing were aligned to V, D, J, and C reference sequence sets of T cell receptors to generate a (library number.vdjca) file.

CDR3 clonotypes were assembled using the result (library number.vdjca) file of the previous step to generate a (library number.clns) file.

Clones and frequencies thereof were derived using the result (library number.clns) file of the previous step to generate a (library number.txt) file.

A NILILa (Non-Invasive Lesions Infiltrating Lymphocytes Analysis) analysis process comprises the following steps:

1) Supposing the ranking of relative abundance of N TCR/BCR gene clones in plasma constitutes a collection Y(yᵢ≤y₂≤ . . . ≤y_{N}), since other TCR/BCR gene clone libraries in a patient's plasma comes from a normal distribution population, disease-specific TCR/BCR clone sub-libraries released from his disease-associated lymphocytes will cause a skewed distribution of a plasma TCR/BCR clone total library after entering plasma; we assume that a probability density function of the TCR/BCR clone frequency distribution of this sample is cdf: F(Y|θ) wherein θ is the decision parameter set of F; θ can be obtained by solving Equation 1 based on the principle of minimum variance.

Thus, Equation 1 can be described as follows:

$$θ = arg {min}_{θ} {\sum }_{i ∈ Λ}^{ } {[ g ( {y}_{i} ) - g ( {F}^{- 1} ( {F}_{i} \vert θ ) ) ]}^{2} ,$$

wherein A is an index set of Y subset, yᵢ represents a relative frequency of the i^{th} TCR/BCR CDR3, and g is a monotonic function that can be differentiated within the value range of Y. Cdf can just be obtained by solving an equation of which the expression is as follows:

$$\frac{1}{2} + \frac{1}{2} erf \{ \frac{{( y - μ )}^{2}}{\sqrt{2} σ} \} ,$$

wherein erf is an error function, y is a clone frequency value, μ is a frequency mean and σ is a standard deviation. A TCR/BCR clone frequency distribution detected in plasma can be solved according to this model probability density distribution function. Supposing there are two thresholds [symbol] _{ρ}^{±}, when a frequency of TCR/BCR is higher than [symbol] _{ρ}⁺ or lower than [symbol] _{ρ}⁻, the number of CDR3 is ρ_{±}, and then Equation 2 can be solved, the expression of Equation 2 being as follows:

$${𝓁}_{ρ}^{\pm } = {F}^{- 1} ( {δ}_{\pm } ∓ \frac{{ρ}_{\pm }}{N} \vert θ ) ,$$

the expression of a threshold [symbol] _{ρ}^{±} can just be obtained as follows:

$${𝓁}_{ρ}^{\pm } = \sqrt{2} σ {erf}^{- 1} [ \pm ( 1 - 2 \frac{{ρ}_{\pm }}{N} ) ] + μ .$$

2) In order to explore more outlier TCR/BCR gene clones associated with lesion sites, we set ρ_{±} to 1. Thus, the relative frequency value ?_{ρ}^{±} characterizing the outlier TCR/BCR gene clones can be calculated, and this value can be used as the boundary of distinguishing outliers, and a frequency value corresponding to this point is called plasma B (boundary, B) point.

The B point values of three cases were calculated according to the method shown above, the specific results being shown in Table 4.

TABLE 4
Plasma B point values of calculation for 3 cases

| Case | Lymphocyte | Library | Plasma B point |
|---|---|---|---|
| No. | subpopulation | No. | value |
| Case 1 | Plasma cf-DNA | Lab-A-1 | 0.000123 |
| Case 2 | Plasma cf-DNA | Lab-B-1 | 0.000114 |
| Case 3 | Plasma cf-DNA | Lab-C-1 | 0.000179 |

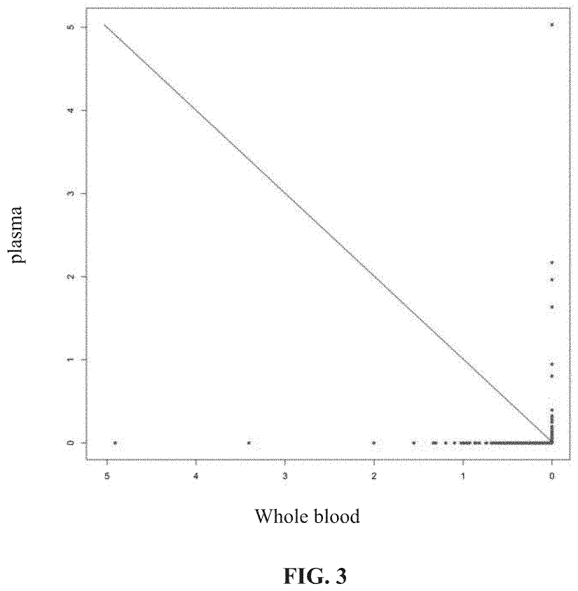

In order to further avoid an impact of a lymphocyte total library in PBMCs on results, the filtering method shown in FIG. 3 was carried out: drawing a coordinate chart in which an abscissa is an order of a frequency of clones detected in the PBMCs from high to low, and an ordinate is an order of a frequency of clones detected in the plasma from low to high; in this chart, frequency coordinates of each clone in the two samples are marked, and then two points are found: abscissa and ordinate values of the first point are both maximum values, and an abscissa value of the second point is a minimum value and an ordinate value is B value; these two points are connected to form a straight line which divides coordinates into two parts: the upper right part is a distribution area of LILs, and the lower left part is a distribution area of other background clones. Points in the upper right part are output, and are just CDR3 sequences of the LILs. After statistics, 65 CDR3 sequences were screened out in 3 cases, and the detailed results are shown in Table 5.

TABLE 5
Number of CDR3 sequences obtained from analysis of 3 cases

| | Plasma B point | Number of |
|---|---|---|
| Case No. | value | CDR3 sequences |
| Case 1 | 0.000126 | 25 |
| Case 2 | 0.000114 | 16 |
| Case 3 | 0.000179 | 24 |

5) Tumor tissue samples from 3 cases were detected; the analysis of tumor tissue TCR detection revealed that the proportion of tumor lesions infiltrating lymphocytes detected in peripheral blood samples was more than 80% after NILILa analysis (see Table 6).

TABLE 6
Number of CDR3 sequences obtained from analysis of 3 cases

| | Number of | | | Proportion |
|---|---|---|---|---|
| | CDR3 | | | of CDR3 |
| | sequences | | | shared by |
| | obtained | Number of | | two CDR3 |
| | from | CDR3 sequences | Number of CDR3 | sequences |
| Case | NILILa | actually detected | shared by two | in NILILa |
| No. | analysis | in tumor tissues | CDR3 sequences | analysis |
| Case 1 | 25 | 78 | 20 | 80.0% |
| Case 2 | 16 | 56 | 14 | 87.5% |
| Case 3 | 24 | 81 | 20 | 83.3% |

For the CDR3 sequences obtained by the NILILa assay, results can be reported as normal and abnormal results by, for example, determining the percentage of total clones detected in patients' samples, or comparing normal ranges with numbers and sequence structures of individual patients obtained. This provides physicians with additional clinical testing for diagnostic purposes.

1. A system for assessing immune repertoire diversity of a T cell antigen receptor (TCR) or a B cell antigen receptor (BCR) in a plasma cell-free DNA (cf-DNA) and a nuclear DNA (g-DNA) isolated from peripheral blood mononuclear cells (PBMCs), comprising the following units:
1) a reference sequence set construction unit that constructs a reference sequence set;
2) a sample preparation unit;
3) a library preparation and high-throughput sequencing unit; and
4) a immune repertoire bioinformatics analysis unit.

2. The system according to claim 1, wherein the reference sequence set is a specific amplification primer designed according to the sequence of the TCR, and an immune repertoire sequence set is thus constructed according to an amplified fragment of the TCR amplified using the specific amplification primer; or wherein the reference sequence set is a specific amplification primer designed according to the sequence of the BCR, and an immune repertoire sequence set is thus constructed according to an amplified fragment of the BCR amplified using the specific amplification primer.

3. The system according to claim 1, wherein the sample preparation unit prepares a sample by a process comprising the following steps:
1) separating PBMCs from peripheral blood of a subject to be tested;
2) extracting the cf-DNA from a plasma sample of the peripheral blood, and extracting the nuclear DNA (g-DNA) from the PBMCs sample; and
3) determining DNA quality of the cf-DNA and the nuclear DNA.

4. The system according to claim 1, wherein the library preparation and high-throughput sequencing unit carries out a process comprising the following steps:
1) subjecting the cf-DNA and the gDNA to multiplex PCR amplification of a CDR3 sequence of a TCR β chain, or subjecting the cf-DNA and the gDNA to multiplex PCR amplification of a CDR3 sequence of a BCR H chain respectively;
2) purifying a first amplification product of the previous step;
3) further amplifying a target fragment of the first amplification product using a library linker primer;
4) performing purification and fragment selection on a second amplification product of the target fragment to obtain a library of amplified product from the second amplification product; and
5) performing sequencing of the library using a high-throughput sequencer.

5. The system according to claim 1, wherein the bioinformatics analysis unit is capable of executing the following instructions:
1) performing MiXCR software analysis, filtering out low-quality data, correcting PCR and sequencing errors, and identifying CDR3 sequences;
2) performing non-invasive lesions infiltrating lymphocytes analysis (NILILa), comprising the following contents:
if the ranking of relative abundance of N TCRs/BCRs in plasma constitutes a collection Y(y₁≤y₂≤ . . . ≤y_{N}), since a normal TCR/BCR library in a patient's plasma comes from a normal distribution population, the disease-specific TCR/BCR sub-library released from his lesion sites will cause a skewed distribution of a plasma TCR/BCR total library after entering plasma; supposing the probability density function of the skewed distribution is cdf: F (Y|θ), wherein θ is the decision parameter set of F; θ can be obtained by solving Equation 1 based on the principle of minimum variance, Equation 1 being described as follows:
$$θ = arg {min}_{θ} {\sum }_{i ∈ Λ}^{ } {[ g ( {y}_{i} ) - g ( {F}^{- 1} ( {F}_{i} \vert θ ) ) ]}^{2} ,$$
wherein A is an index set of Y subset, yᵢ represents a relative abundance of the i^{th} TCR/BCR CDR3, g is a monotonic function that can be differentiated within the value range of Y; cdf is obtained by solving this equation, the expression of cdf being as follows:
$$\frac{1}{2} + \frac{1}{2} erf \{ \frac{{( y - μ )}^{2}}{\sqrt{2} σ} \} ,$$
a TCR/BCR frequency distribution detected in plasma can be determined according to this model probability density distribution function; supposing there are two thresholds [symbol] _{ρ}^{±}, when a frequency of TCR/BCR is higher than [symbol] _{ρ}⁺ or lower than [symbol] _{ρ}⁻, the number of CDR3 is ρ_{±}, and Equation 2 is solved, the expression of Equation 2 being as follows:
$${𝓁}_{ρ}^{\pm } = {F}^{- 1} ( {δ}_{\pm } ∓ \frac{{ρ}_{\pm }}{N} \vert θ ) ,$$
a threshold [symbol] _{ρ}^{±} is obtained as follows:
$${𝓁}_{ρ}^{\pm } = \sqrt{2} σ {erf}^{- 1} [ \pm ( 1 - 2 \frac{{ρ}_{\pm }}{N} ) ] + μ ,$$
furthermore, in order to explore more outlier TCRs/BCRs associated with lesion sites, ρ_{±} is set to 1, a relative abundance value [symbol] _{ρ}^{±} characterizing outlier TCRs/BCRs is calculated, and then this value can be used as a boundary of distinguishing outliers, and a frequency value corresponding to this point is called the plasma B (boundary, B) point;
furthermore, in order to avoid an impact of a lymphocyte total library in PBMCs on results, the following chart is drawn to exclude interference from the lymphocyte total library in PBMCs: an abscissa is an order of a frequency of clones detected in PBMCs from high to low, and an ordinate is an order of a frequency of clones detected in plasma from low to high; in this chart, frequency coordinates of each clone in two samples are marked, and then two points are found: abscissa and ordinate values of the first point are both maximum values, and the second point has an abscissa value of 0 and an ordinate value of B value; these two points are connected to form a line segment which divides coordinates into two parts: the upper right part is a distribution area of lesions infiltrating lymphocytes, and the lower left part is a distribution area of other background clones; points in the upper right part are output, and are CDR3 sequences of lesions infiltrating lymphocytes.

6-8. (canceled)

9. A primer combination for detecting TCR or BCR immune repertoire in plasma cfDNA and PMBC gDNA, wherein the sequences of the primer combination are shown in the FIG. 1 and FIG. 2.

10. A kit for detecting TCR immune repertoire in plasma cfDNA and PMBC gDNA, comprising the primer combination according to claim 9.

11. A bioinformatics analysis unit, capable of executing the following instructions:
1) performing MiXCR software analysis, filtering out low-quality data, correcting PCR and sequencing errors, and identifying CDR3 sequences;
2) performing non-invasive lesions infiltrating lymphocytes analysis (NILILa), comprising the following contents:
if the ranking of relative abundance of N TCRs/BCRs in plasma constitutes a collection Y(y₁≤y₂≤ . . . ≤y_{N}), since a normal TCR/BCR library in a patient's plasma comes from a normal distribution population, the disease-specific TCR/BCR sub-library released from his lesion sites will cause a skewed distribution of a plasma TCR/BCR total library after entering plasma; supposing the probability density function of the skewed distribution is cdf: F (Y|θ), wherein θ is the decision parameter set of F; θ can be obtained by solving Equation 1 based on the principle of minimum variance, Equation 1 being described as follows:
$$θ = arg {min}_{θ} {\sum }_{i ∈ Λ}^{ } {[ g ( {y}_{i} ) - g ( {F}^{- 1} ( {F}_{i} \vert θ ) ) ]}^{2} ,$$
wherein A is an index set of Y subset, represents a relative abundance of the i^{th} TCR/BCR CDR3, g is a monotonic function that can be differentiated within the value range of Y; cdf is obtained by solving this equation, the expression of cdf being as follows:
$$\frac{1}{2} + \frac{1}{2} erf \{ \frac{{( y - μ )}^{2}}{\sqrt{2} σ} \} ,$$
a TCR/BCR frequency distribution detected in plasma can be determined according to this model probability density distribution function; supposing there are two thresholds [symbol] _{ρ}^{±}, when a frequency of TCR/BCR is higher than [symbol] _{ρ}⁺ or lower than [symbol] _{ρ}⁻, the number of CDR3 is ρ_{±}, and Equation 2 is solved, the expression of Equation 2 being as follows:
$${𝓁}_{ρ}^{\pm } = {F}^{- 1} ( {δ}_{\pm } ∓ \frac{{ρ}_{\pm }}{N} \vert θ ) ,$$
a threshold [symbol] _{ρ}^{±} is obtained as follows:
$${𝓁}_{ρ}^{\pm } = \sqrt{2} σ {erf}^{- 1} [ \pm ( 1 - 2 \frac{{ρ}_{\pm }}{N} ) ] + μ ,$$
furthermore, in order to explore more outlier TCRs/BCRs associated with lesion sites, ρ_{±} is set to 1, a relative abundance value [symbol] _{ρ}^{±} characterizing outlier TCRs/BCRs is calculated, and then this value can be used as a boundary of distinguishing outliers, and a frequency value corresponding to this point is called the plasma B (boundary, B) point;
furthermore, in order to avoid an impact of a lymphocyte total library in PBMCs on results, the following chart is drawn to exclude interference from the lymphocyte total library in PBMCs: an abscissa is an order of a frequency of clones detected in PBMCs from high to low, and an ordinate is an order of a frequency of clones detected in plasma from low to high; in this chart, frequency coordinates of each clone in two samples are marked, and then two points are found: abscissa and ordinate values of the first point are both maximum values, and the second point has an abscissa value of 0 and an ordinate value of B value; these two points are connected to form a line segment which divides coordinates into two parts: the upper right part is a distribution area of lesions infiltrating lymphocytes, and the lower left part is a distribution area of other background clones; points in the upper right part are output, and are CDR3 sequences of lesions infiltrating lymphocytes.

12. A method for analyzing in a subject in need thereof immune repertoire diversity of TCRs or BCRs in plasma cell-free DNA (cf-DNA) and nuclear DNA (g-DNA) isolated from peripheral blood mononuclear cells (PBMCs) of said subject, the method comprising the following steps:
1) Constructing a reference sequence set and designing a specific amplification primer;
2) preparing the cf-DNA and the g-DNA from peripheral blood of the subject;
3) preparing and sequencing a library of PCR amplification products of the cf-DNA and the g-DNA to obtain sequence data; and
4) Analyzing bioinformatics based on the sequence data obtained in 3).

13. The method according to claim 12, wherein step 1) is carried out based on a TCR or BCR reference sequence.

14. The method according to claim 12, wherein step 2) comprises the following steps:
1) separating PBMCs from peripheral blood of the subject;
2) extracting cf-DNA from a plasma sample of the peripheral blood, and extracting nuclear DNA from the PBMCs; and
3) determining DNA quality of the cf-DNA and the nuclear DNA.

15. The method according to claim 12, wherein step 3) comprises the following steps:
1) subjecting the cf-DNA and the gDNA to multiplex PCR amplification of a CDR3 sequence of a TCR β chain; or subjecting the cf-DNA and the gDNA to multiplex PCR amplification of a CDR3 sequence of a BCR H chain;
2) purifying a first amplification product of the previous step;
3) further amplifying a target fragment of the first amplification product using a library linker primer;
4) performing purification and fragment selection on a second amplification product of the target fragment to obtain a library of amplified product from the second amplification product; and
5) performing sequencing of the library using a high-throughput sequencer.

16. The method according to claim 12, wherein step 4) comprises executing the following instructions:
1) performing MiXCR software analysis, filtering out low-quality data, correcting PCR and sequencing errors, and identifying CDR3 sequences;
2) performing non-invasive lesions infiltrating lymphocytes analysis (NILILa), comprising the following contents:
if the ranking of relative abundance of N TCRs/BCRs in plasma constitutes a collection Y(y₁≤y₂≤ . . . ≤y_{N}), since a normal TCR/BCR library in a patient's plasma comes from a normal distribution population, the disease-specific TCR/BCR sub-library released from his lesion sites will cause a skewed distribution of a plasma TCR/BCR total library after entering plasma; supposing the probability density function of the skewed distribution is cdf: F (Y|θ), wherein θ is the decision parameter set of F; θ can be obtained by solving Equation 1 based on the principle of minimum variance, Equation 1 being described as follows:
$$θ = arg {min}_{θ} {\sum }_{i ∈ Λ}^{ } {[ g ( {y}_{i} ) - g ( {F}^{- 1} ( {F}_{i} \vert θ ) ) ]}^{2} ,$$
wherein A is an index set of Y subset, yᵢ represents a relative abundance of the i^{th} TCR/BCR CDR3, g is a monotonic function that can be differentiated within the value range of Y; cdf is obtained by solving this equation, the expression of cdf being as follows:
$$\frac{1}{2} + \frac{1}{2} erf \{ \frac{{( y - μ )}^{2}}{\sqrt{2} σ} \} ,$$
a TCR/BCR frequency distribution detected in plasma can be determined according to this model probability density distribution function; supposing there are two thresholds [symbol] _{ρ}^{±}, when a frequency of TCR/BCR is higher than [symbol] _{ρ}⁺or lower than [symbol] _{ρ}⁻, the number of CDR3 is ρ_{±}, and Equation 2 is solved, the expression of Equation 2 being as follows:
$${𝓁}_{ρ}^{\pm } = {F}^{- 1} ( {δ}_{\pm } ∓ \frac{{ρ}_{\pm }}{N} \vert θ ) ,$$
a threshold [symbol] _{ρ}^{±} is obtained as follows:
$${𝓁}_{ρ}^{\pm } = \sqrt{2} σ {erf}^{- 1} [ \pm ( 1 - 2 \frac{{ρ}_{\pm }}{N} ) ] + μ ,$$
furthermore, in order to explore more outlier TCRs/BCRs associated with lesion sites, ρ_{±} is set to 1, a relative abundance value [symbol] _{ρ}^{±} characterizing outlier TCRs/BCRs is calculated, and then this value can be used as a boundary of distinguishing outliers, and a frequency value corresponding to this point is called the plasma B (boundary, B) point;
furthermore, in order to avoid an impact of a lymphocyte total library in PBMCs on results, the following chart is drawn to exclude interference from the lymphocyte total library in PBMCs: an abscissa is an order of a frequency of clones detected in PBMCs from high to low, and an ordinate is an order of a frequency of clones detected in plasma from low to high; in this chart, frequency coordinates of each clone in two samples are marked, and then two points are found: abscissa and ordinate values of the first point are both maximum values, and the second point has an abscissa value of 0 and an ordinate value of B value; these two points are connected to form a line segment which divides coordinates into two parts: the upper right part is a distribution area of lesions infiltrating lymphocytes, and the lower left part is a distribution area of other background clones; points in the upper right part are output, and are CDR3 sequences of lesions infiltrating lymphocytes.

17. A method for screening or identifying lesions infiltrating lymphocytes, comprising using the system according to claim 1.

18. A method for diagnosing or screening a disease, comprising using the system according to claim 1.

19. The method according to claim 18, characterized in that the disease is selected from the group consisting of tumor, autoimmune disease, and infectious disease.

20. The method according to claim 18, wherein the system comprises a reference sequence set construction unit and the reference sequence set is a specific amplification primer designed according to the sequence of the TCR or BCR, and an immune repertoire sequence set is thus constructed according to an amplified fragment.
